(19) United States
(12) Patent Application Publication
(10) Pub. No.: US 2026/0227470 A1
(43) Pub. Date: Aug. 6, 2026

(54) SYSTEM AND METHOD FOR DEEP LEARNING-BASED ULTRAFAST AND ROBUST RF SHIMMING USING QUADRATURE B1+ MAPS

(71) Applicant: GE Precision Healthcare LLC, Waukesha, WI (US)

(72) Inventors: Xiaoxuan He, Milwaukee, WI (US); Baolian Yang, Pewaukee, WI (US); Eric Michael Printz, Oconomowoc, WI (US); Gaohong Wu, New Berlin, WI (US); Ling Sun, Brookfield, WI (US); Daming Shen, West Allis, WI (US)

(21) Appl. No.: 19/045,021

(22) Filed: Feb. 4, 2025

(51) Int. Cl. G01R 33/34; A61B 5/00; A61B 5/055; G01R 33/38; G01R 33/58

(52) U.S. Cl. CPC G01R 33/34; A61B 5/0033; A61B 5/055; G01R 33/38; G01R 33/58

(57) ABSTRACT

A computer-implemented method for performing radio frequency shimming includes obtaining, via a processing system including one or more processors, quadrature B1+ maps of an imaging volume of a subject. The computer-implemented method also includes inputting, via the processing system, the quadrature B1+ maps into a deep learning-based radio frequency shimming inference model. The computer-implemented method further includes outputting, via the processing system, from the deep learning-based radio frequency shimming inference model radio frequency shims based on the quadrature B1+ maps. The computer-implemented method even further includes applying, via the processing system, the radio frequency shims to a magnetic resonance imaging scanner prior to a parallel transmission diagnostic scan of the imaging volume of the subject.

### BACKGROUND

The subject matter disclosed herein relates to medical imaging and, more particularly, to a system and a method for deep learning-based ultrafast and robust radio frequency (RF) shimming using quadrature B1+ maps.

Non-invasive imaging technologies allow images of the internal structures or features of a patient/object to be obtained without performing an invasive procedure on the patient/object. In particular, such non-invasive imaging technologies rely on various physical principles (such as the differential transmission of X-rays through a target volume, the reflection of acoustic waves within the volume, the paramagnetic properties of different tissues and materials within the volume, the breakdown of targeted radionuclides within the body, and so forth) to acquire data and to construct images or otherwise represent the observed internal features of the patient/object.

During MRI, when a substance such as human tissue is subjected to a uniform magnetic field (polarizing field B₀), the individual magnetic moments of the spins in the tissue attempt to align with this polarizing field, but precess about it in random order at their characteristic Larmor frequency. If the substance, or tissue, is subjected to a magnetic field (excitation field B₁) which is in the x-y plane and which is near the Larmor frequency, the net aligned moment, or “longitudinal magnetization”, M_{z}, may be rotated, or “tipped”, into the x-y plane to produce a net transverse magnetic moment, Mₜ. A signal is emitted by the excited spins after the excitation signal B₁ is terminated and this signal may be received and processed to form an image.

When utilizing these signals to produce images, magnetic field gradients (Gₓ, G_{y}, and G_{z}) are employed. Typically, the region to be imaged is scanned by a sequence of measurement cycles in which these gradient fields vary according to the particular localization method being used. The resulting set of received nuclear magnetic resonance (NMR) signals are digitized and processed to reconstruct the image using one of many well-known reconstruction techniques.

RF shimming is a widely used technique to improve MRI quality by adjusting the phase and the magnitude of the transmits channels in the RF coil and to eliminate field inhomogeneity. RF shimming is commonly used at ultrahigh fields to mitigate B1+ inhomogeneities. However, it requires a time consuming per-channel B1+ mapping scan, which can be sensitive to motion, potentially compromising RF shim accuracy and image quality. In addition, additional optimization strategies are often needed to address the local minima problem.

### BRIEF DESCRIPTION

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

In one embodiment, a computer-implemented method for performing radio frequency shimming is provided. The computer-implemented method includes obtaining, via a processing system including one or more processors, quadrature B1+ maps of an imaging volume of a subject. The computer-implemented method also includes inputting, via the processing system, the quadrature B1+ maps into a deep learning-based radio frequency shimming inference model. The computer-implemented method further includes outputting, via the processing system, from the deep learning-based radio frequency shimming inference model radio frequency shims based on the quadrature B1+ maps. The computer-implemented method even further includes applying, via the processing system, the radio frequency shims to a magnetic resonance imaging scanner prior to a parallel transmission diagnostic scan of the imaging volume of the subject.

In another embodiment, a system for performing radio frequency shimming is provided. The system includes a memory encoding processor-executable routines. The system also includes a processing system including one or more processors and configured to access the memory and to execute the processor-executable routines, wherein the processor-executable routines, when executed by the processing system, cause the processing system to perform actions. The actions include obtaining quadrature B1+ maps of an imaging volume of a subject. The actions also include inputting the quadrature B1+ maps into a deep learning-based radio frequency shimming inference model. The actions further include outputting from the deep learning-based radio frequency shimming inference model radio frequency shims based on the quadrature B1+ maps. The actions even further include applying the radio frequency shims to a magnetic resonance imaging scanner prior to a parallel transmission diagnostic scan of the imaging volume of the subject.

In a further embodiment, a non-transitory computer-readable medium, the computer-readable medium including processor-executable code that when executed by a processing system including one or more processors, causes the processing system to perform actions. The actions include obtaining quadrature B1+ maps of an imaging volume of a subject. The actions also include inputting the quadrature B1+ maps into a deep learning-based radio frequency shimming inference model. The actions further include outputting from the deep learning-based radio frequency shimming inference model radio frequency shims based on the quadrature B1+ maps. The actions even further include applying the radio frequency shims to a magnetic resonance imaging scanner prior to a parallel transmission diagnostic scan of the imaging volume of the subject.

### BRIEF DESCRIPTION OF THE DRAWINGS

These and other features, aspects, and advantages of the present subject matter will become better understood when the following detailed description is read with reference to the accompanying drawings in which like characters represent like parts throughout the drawings, wherein:

FIG. 1 illustrates an embodiment of a magnetic resonance imaging (MRI) system suitable for use with the disclosed technique, in accordance with aspects of the present disclosure;

FIG. 2 illustrates a flow diagram of a method for performing radio frequency shimming, in accordance with aspects of the present disclosure;

FIG. 3 illustrates a flow diagram of method for performing radio frequency shimming (e.g., utilizing acquired quadrature B1+ maps), in accordance with aspects of the present disclosure;

FIG. 4 illustrates a flow diagram of method for performing radio frequency shimming (e.g., utilizing localizer images), in accordance with aspects of the present disclosure;

FIG. 5 illustrates a schematic diagram of a workflow for performing radio frequency shimming, in accordance with aspects of the present disclosure;

FIG. 6 illustrates a schematic diagram illustrating utilization of a trained neural network for performing radio frequency shimming, in accordance with aspects of the present disclosure;

FIG. 7 depicts the performance of the deep learning-based radio frequency shimming inference model when B1+ maps are corrupted by motion, in accordance with aspects of the present disclosure;

FIG. 8 depicts the performance of the deep learning-based radio frequency shimming inference model in a repeated RF shim scan, in accordance with aspects of the present disclosure;

FIG. 9 illustrates a performance of the deep learning-based radio frequency shimming inference model with respect to low B1+ regions, in accordance with aspects of the present disclosure;

FIG. 10 illustrates a training performance of the deep learning-based radio frequency shimming inference model, in accordance with aspects of the present disclosure;

FIG. 11 illustrates a testing performance of the deep learning-based radio frequency shimming inference model, in accordance with aspects of the present disclosure;

FIG. 12 depicts MR images demonstrating a performance of the deep learning-based radio frequency shimming inference model on a previously seen subject, in accordance with aspects of the present disclosure;

FIG. 13 depicts MR images demonstrating a performance of the deep learning-based radio frequency shimming inference model on a previously unseen subject, in accordance with aspects of the present disclosure; and

FIG. 14 depicts MR images demonstrating a performance of the deep learning-based radio frequency shimming inference model on an additional previously unseen subject, in accordance with aspects of the present disclosure.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers'specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present subject matter, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

While aspects of the following discussion are provided in the context of medical imaging, it should be appreciated that the disclosed techniques are not limited to such medical contexts. Indeed, the provision of examples and explanations in such a medical context is only to facilitate explanation by providing instances of real-world implementations and applications. However, the disclosed techniques may also be utilized in other contexts, such as image reconstruction for non-destructive inspection of manufactured parts or goods (i.e., quality control or quality review applications), and/or the non-invasive inspection of packages, boxes, luggage, and so forth (i.e., security or screening applications). In general, the disclosed techniques may be useful in any imaging or screening context or image processing or photography field where a set or type of acquired data undergoes a reconstruction process to generate an image or volume.

Deep-learning (DL) approaches discussed herein may be based on artificial neural networks, and may therefore encompass one or more of deep neural networks, fully connected networks, convolutional neural networks (CNNs), unrolled neural networks, perceptrons, encoders-decoders, recurrent networks, wavelet filter banks, u-nets, general adversarial networks (GANs), dense neural networks, or other neural network architectures. The neural networks may include shortcuts, activations, batch-normalization layers, and/or other features. These techniques are referred to herein as DL techniques, though this terminology may also be used specifically in reference to the use of deep neural networks, which is a neural network having a plurality of layers.

As discussed herein, DL techniques (which may also be known as deep machine learning, hierarchical learning, or deep structured learning) are a branch of machine learning techniques that employ mathematical representations of data and artificial neural networks for learning and processing such representations. By way of example, DL approaches may be characterized by their use of one or more algorithms to extract or model high level abstractions of a type of data-of-interest. This may be accomplished using one or more processing layers, with each layer typically corresponding to a different level of abstraction and, therefore potentially employing or utilizing different aspects of the initial data or outputs of a preceding layer (i.e., a hierarchy or cascade of layers) as the target of the processes or algorithms of a given layer. In an image processing or reconstruction context, this may be characterized as different layers corresponding to the different feature levels or resolution in the data. In general, the processing from one representation space to the next-level representation space can be considered as one ‘stage’ of the process. Each stage of the process can be performed by separate neural networks or by different parts of one larger neural network.

The present disclosure provides systems and methods for performing deep learning-based radio frequency shimming. The disclosed technique is based on the observation that a subject-specific B1+ pattern in the quadrature B1+ maps can empirically predict how well RF shimming will work for the subject, and there are a few but limited patterns across subjects, and the relationship between these patterns and their corresponding optimal RF shim can be learned by a neural network. In particular, disclosed systems and methods utilize a calibration scan to map all-channel combined (quadrature) B1+ (as opposed to performing a calibration scan to get per-channel B1+ maps). The disclosed systems and methods also include calculating RF shims utilizing a neural network that is trained on all-channel combined (quadrature) B1+ (as opposed to calculating RF shims using per-channel B1+ data). The disclosed systems and methods provide a faster and more robust RF shimming workflow to mitigate B1+ non-uniformities at ultrahigh field and improve image quality. The deep learning based RF shimming provides a faster workflow for ultrahigh field imaging. B1+ calibration scan time is greatly reduced with less sensitivity to motion while providing more consistent improvements in image quality. For example, a parallel transmission calibration scan may be reduced by 8 to 16 times (i.e., which scales with the number of transmitters). For example, for a 7 Tesla (T) MRI scanner, calibration scan time can be reduced from one minute (or longer) to less than ten seconds. The saved time builds up substantially as multiple calibration scans are often needed in one exam. In addition, the neural network memorizes empirical B1+ map data and makes less mistakes when there is motion corruption. Due to the deep learning trained model yielding a more consistent and robust RF shim, it helps deliver higher quality images more reliably. The disclosed technique benefits ultrahigh field MRI scanners (5 T, 7 T, and above) that are all equipped with at least 8-channel parallel transmission system. Even though the technique is most beneficial for ultrahigh field MRI scanners, it can be used for 3 T MRI scanners and above having at least two transmit channels.

The disclosed embodiments include providing a system and a method for performing radio frequency shimming. The system and the method include obtaining, via a processing system including one or more processors, quadrature B1+ maps of an imaging volume of a subject. The system and the method also include inputting, via the processing system, the quadrature B1+ maps into a deep learning-based radio frequency shimming inference model. The system and the method further include outputting, via the processing system, from the deep learning-based radio frequency shimming inference model radio frequency shims based on the quadrature B1+ maps. The system and the method even further include applying, via the processing system, the radio frequency shims to a magnetic resonance imaging scanner prior to a parallel transmission diagnostic scan of the imaging volume of the subject.

In certain embodiments, the system and the method include receiving, via the processing system, a prescription for the parallel transmission diagnostic scan of the imaging volume of the subject. In certain embodiments, the system and the method include, after receiving the prescription, performing, via the processing system, a parallel transmission calibration scan of the imaging volume of the subject with the magnetic resonance imaging scanner to obtain the quadrature B1+ maps by mapping all transmit channels combined. In certain embodiments, the system and the method include, after receiving the prescription, acquiring, via the processing system, localizer images of the imaging volume of the subject utilizing the magnetic resonance imaging scanner; and synthesizing, via the processing system, the quadrature B1+ maps based on the localizer images.

In certain embodiments, the system and the method include performing, via the processing system, the parallel transmission diagnostic scan to acquire magnetic resonance scan data of the imaging volume of the subject. In certain embodiments, the system and the method include generating, via the processing system, a reconstructed image based on the magnetic resonance scan data. In certain embodiments, the magnetic resonance imaging scanner includes at least two transmit channels and the magnetic resonance imaging scanner has a magnetic field strength of at least 3 Tesla.

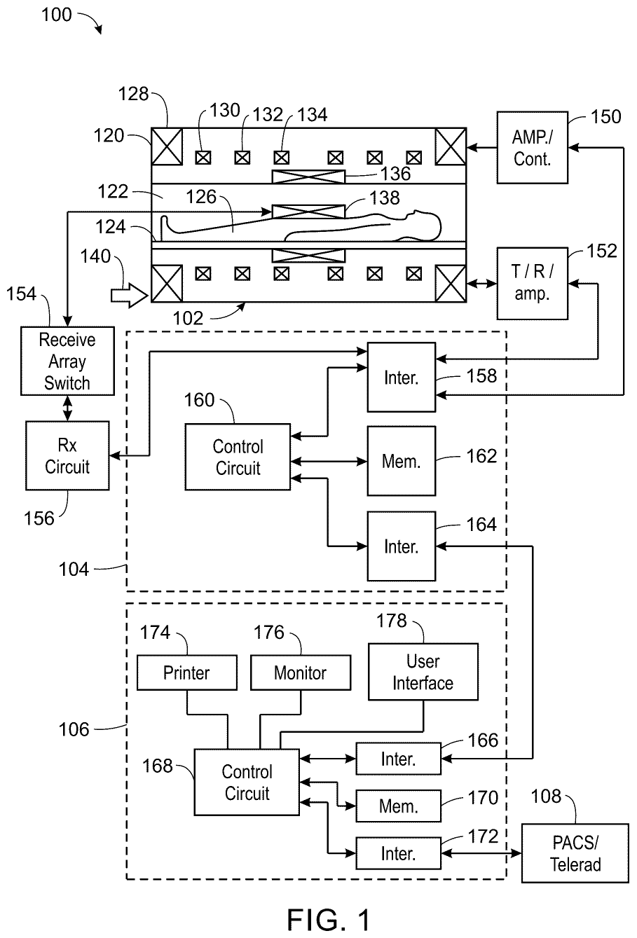

With the preceding in mind, FIG. 1 a magnetic resonance imaging (MRI) system 100 is illustrated schematically as including a scanner 102, scanner control circuitry 104, and system control circuitry 106. According to the embodiments described herein, the MRI system 100 is generally configured to perform MR imaging.

System 100 additionally includes remote access and storage systems or devices such as picture archiving and communication systems (PACS) 108, or other devices such as teleradiology equipment so that data acquired by the system 100 may be accessed on-or off-site. In this way, MR data may be acquired, followed by on-or off-site processing and evaluation. While the MRI system 100 may include any suitable scanner or detector, in the illustrated embodiment, the system 100 includes a full body scanner 102 having a housing 120 through which a bore 122 is formed. A table 124 is moveable into the bore 122 to permit a patient 126 (e.g., subject) to be positioned therein for imaging selected anatomy within the patient.

Scanner 102 includes a series of associated coils for producing controlled magnetic fields for exciting the gyromagnetic material within the anatomy of the patient being imaged. Specifically, a primary magnet coil 128 is provided for generating a primary magnetic field, B₀, which is generally aligned with the bore 122. A series of gradient coils 130, 132, and 134 permit controlled magnetic gradient fields to be generated for positional encoding of certain gyromagnetic nuclei within the patient 126 during examination sequences. A radio frequency (RF) coil 136 (e.g., RF transmit coil) is configured to generate radio frequency pulses for exciting the certain gyromagnetic nuclei within the patient. In addition to the coils that may be local to the scanner 102, the system 100 also includes a set of receiving coils or RF receiving coils 138 (e.g., an array of coils) configured for placement proximal (e.g., against) to the patient 126. As an example, the receiving coils 138 can include cervical/thoracic/lumbar (CTL) coils, head coils, single-sided spine coils, and so forth. Generally, the receiving coils 138 are placed close to or on top of the patient 126 so as to receive the weak RF signals (weak relative to the transmitted pulses generated by the scanner coils) that are generated by certain gyromagnetic nuclei within the patient 126 as they return to their relaxed state.

The various coils of system 100 are controlled by external circuitry to generate the desired field and pulses, and to read emissions from the gyromagnetic material in a controlled manner. In the illustrated embodiment, a main power supply 140 provides power to the primary field coil 128 to generate the primary magnetic field, Bo. A power input (e.g., power from a utility or grid), a power distribution unit (PDU), a power supply (PS), and a driver circuit 150 may together provide power to pulse the gradient field coils 130, 132, and 134. The driver circuit 150 may include amplification and control circuitry for supplying current to the coils as defined by digitized pulse sequences output by the scanner control circuitry 104.

Another control circuit 152 is provided for regulating operation of the RF coil 136. Circuit 152 includes a switching device for alternating between the active and inactive modes of operation, wherein the RF coil 136 transmits and does not transmit signals, respectively. Circuit 152 also includes amplification circuitry configured to generate the RF pulses. Similarly, the receiving coils 138 are connected to switch 154, which is capable of switching the receiving coils 138 between receiving and non-receiving modes. Thus, the receiving coils 138 resonate with the RF signals produced by relaxing gyromagnetic nuclei from within the patient 126 while in the receiving mode, and they do not resonate with RF energy from the transmitting coils (i.e., coil 136) so as to prevent undesirable operation while in the non-receiving mode. Additionally, a receiving circuit 156 is configured to receive the data detected by the receiving coils 138 and may include one or more multiplexing and/or amplification circuits.

It should be noted that while the scanner 102 and the control/amplification circuitry described above are illustrated as being coupled by a single line, many such lines may be present in an actual instantiation. For example, separate lines may be used for control, data communication, power transmission, and so on. Further, suitable hardware may be disposed along each type of line for the proper handling of the data and current/voltage. Indeed, various filters, digitizers, and processors may be disposed between the scanner and either or both of the scanner and system control circuitry 104, 106.

As illustrated, scanner control circuitry 104 includes an interface circuit 158, which outputs signals for driving the gradient field coils and the RF coil and for receiving the data representative of the magnetic resonance signals produced in examination sequences. The interface circuit 158 is coupled to a control and analysis circuit 160. The control and analysis circuit 160 executes the commands for driving the circuit 150 and circuit 152 based on defined protocols selected via system control circuit 106.

Control and analysis circuit 160 also serves to receive the magnetic resonance signals and performs subsequent processing before transmitting the data to system control circuit 106. Scanner control circuit 104 also includes one or more memory circuits 162, which store configuration parameters, pulse sequence descriptions, examination results, and so forth, during operation.

Interface circuit 164 is coupled to the control and analysis circuit 160 for exchanging data between scanner control circuitry 104 and system control circuitry 106. In certain embodiments, the control and analysis circuit 160, while illustrated as a single unit, may include one or more hardware devices. The system control circuit 106 includes an interface circuit 166, which receives data from the scanner control circuitry 104 and transmits data and commands back to the scanner control circuitry 104. The control and analysis circuit 168 may include a CPU in a multi-purpose or application specific computer or workstation. Control and analysis circuit 168 is coupled to a memory circuit 170 to store programming code for operation of the MRI system 100 and to store the processed image data for later reconstruction, display and transmission. The programming code may execute one or more algorithms that, when executed by a processor, are configured to perform reconstruction of acquired data as described below. In certain embodiments, the memory circuit 170 may store one or more neural networks. For example, the neural networks may include a deep learning-based radio frequency shimming inference model trained to infer RF shims from inputted quadrature B1+ maps. In certain embodiments, the techniques disclosed herein may occur on a separate computing device having processing circuitry and memory circuitry.

A processing component (e.g., a microprocessor or processing circuitry) and a memory of the magnetic resonance imaging system 100, such as may be present in scanner control circuitry 104 and/or system control circuitry 106, may be used to execute stored software code, instructions, or routines for acquiring and processing the MR data. The term “code” or “software code” used herein refers to any instructions or set of instructions that control the magnetic resonance imaging system 100. The code or software code may exist in a computer-executable form, such as machine code, which is the set of instructions and data directly executed by the processing component of the scanner control circuitry 104 and/or system control circuitry 106, human-understandable form, such as source code, which may be compiled in order to be executed by the processing component of the scanner control circuitry 104 and/or system control circuitry 106, or an intermediate form, such as object code, which is produced by a compiler. In some embodiments, the magnetic resonance imaging system 100 may include a plurality of controllers.

As an example, the memory may store processor-executable software code or instructions (e.g., firmware or software), which are tangibly stored on a non-transitory computer readable medium. Additionally or alternatively, the memory may store data. As an example, the memory may include a volatile memory, such as random-access memory (RAM), and/or a nonvolatile memory, such as read-only memory (ROM), flash memory, a hard drive, or any other suitable optical, magnetic, or solid-state storage medium, or a combination thereof. Furthermore, processing component may include multiple microprocessors, one or more “general-purpose” microprocessors, one or more special-purpose microprocessors, and/or one or more application specific integrated circuits (ASICS), or some combination thereof. For example, the processing component may include one or more reduced instruction set (RISC) or complex instruction set (CISC) processors. The processing component may include multiple processors, and/or the memory may include multiple memory devices.

In certain embodiments (e.g., for performing deep learning-based RF shimming), the processing component is configured to obtain quadrature B1+ maps of an imaging volume of a subject. The processing component is configured to input the quadrature B1+ maps into a deep learning-based radio frequency shimming inference model. The processing component is configured to output from the deep learning-based radio frequency shimming inference model radio frequency shims based on the quadrature B1+ maps. The processing component is configured to apply the radio frequency shims to a magnetic resonance imaging scanner prior to a parallel transmission diagnostic scan of the imaging volume of the subject.

In certain embodiments, the processing component is configured to receive a prescription for the parallel transmission diagnostic scan of the imaging volume of the subject. In certain embodiments, the processing component is configured, after receiving the prescription, to performing a parallel transmission calibration scan of the imaging volume of the subject with the magnetic resonance imaging scanner to obtain the quadrature B1+ maps by mapping all transmit channels combined. In certain embodiments, the processing component is configured, after receiving the prescription, to acquire localizer images of the imaging volume of the subject utilizing the magnetic resonance imaging scanner; and to synthesize the quadrature B1+ maps based on the localizer images.

In certain embodiments, the processing component is configured to perform the parallel transmission diagnostic scan to acquire magnetic resonance scan data of the imaging volume of the subject. In certain embodiments, the processing component is configured to generate a reconstructed image based on the magnetic resonance scan data. In certain embodiments, the magnetic resonance imaging scanner includes at least two transmit channels and the magnetic resonance imaging scanner has a magnetic field strength of at least 3 Tesla.

An additional interface circuit 172 may be provided for exchanging image data, configuration parameters, and so forth with external system components such as remote access and storage devices 108. Finally, the system control and analysis circuit 168 may be communicatively coupled to various peripheral devices for facilitating operator interface and for producing hard copies of the reconstructed images. In the illustrated embodiment, these peripherals include a printer 174, a monitor 176, and user interface 178 including devices such as a keyboard, a mouse, a touchscreen (e.g., integrated with the monitor 176), and so forth.

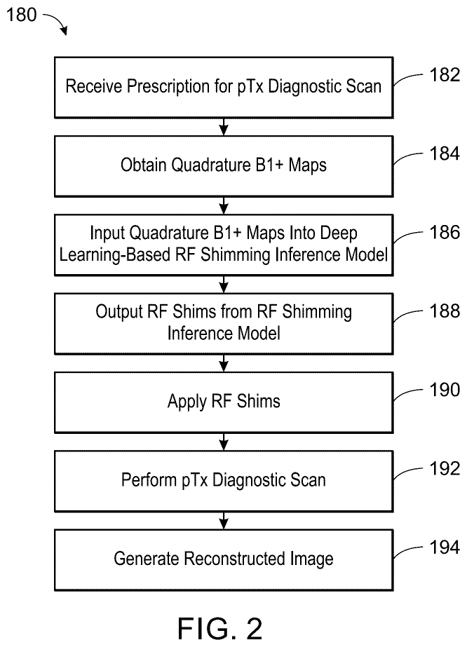

FIG. 2 illustrates a flow diagram of a method 180 for performing radio frequency (RF) shimming. One or more steps of the method 180 may be performed by processing circuitry of the magnetic resonance imaging system 100 in FIG. 1 or a remote computing device. One or more of the steps of the method 180 may be performed simultaneously and/or in a different order from that depicted in FIG. 2. The method 180 is most beneficial for ultrahigh field MRI scanners (5 T, 7 T, and above) that are all equipped with at least 8-channel parallel transmission system. However, the method 180 can also be used for 3 T MRI scanners and above having at least two transmit channels.

The method 180 includes receiving a prescription for the parallel transmission (pTx) diagnostic scan of the imaging volume of the subject (block 182). The prescription is prescribed via user input from a user or operator. The method 180 also includes obtaining quadrature B1+ maps of an imaging volume of a subject (block 184). The method 180 further includes inputting the quadrature B1+ maps into a deep learning-based radio frequency shimming inference model (block 186). The radio frequency shimming inference model is configured to infer RF shims based on inputted quadrature B1+ maps. The method 180 even further includes outputting from the deep learning-based radio frequency shimming inference model radio frequency shims based on the quadrature B1+ maps (block 188). The method 180 further includes applying the radio frequency shims to a magnetic resonance imaging scanner prior to the parallel transmission diagnostic scan of the imaging volume of the subject (block 190). The method 180 further includes performing the parallel transmission diagnostic scan to acquire magnetic resonance scan data of the imaging volume of the subject (block 192). The method 180 even further includes generating a reconstructed image based on the magnetic resonance scan data (block 194).

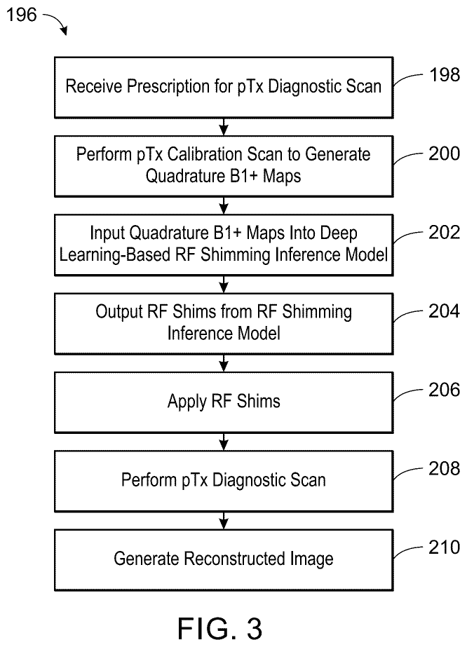

FIG. 3 illustrates a flow diagram of a method 196 for performing radio frequency (RF) shimming (e.g., utilizing acquired quadrature B1+ maps). One or more steps of the method 196 may be performed by processing circuitry of the magnetic resonance imaging system 100 in FIG. 1 or a remote computing device. One or more of the steps of the method 196 may be performed simultaneously and/or in a different order from that depicted in FIG. 3. The method 196 is most beneficial for ultrahigh field MRI scanners (5 T, 7 T , and above) that are all equipped with at least 8-channel parallel transmission system. However, the method 196 can also be used for 3 T MRI scanners and above having at least two transmit channels.

The method 196 includes receiving a prescription for the parallel transmission diagnostic scan of the imaging volume of the subject (block 198). The prescription is prescribed via user input from a user or operator. The method 196 also includes, after receiving the prescription, performing a parallel transmission calibration scan of the imaging volume of the subject with the magnetic resonance imaging scanner to generate quadrature B1+ maps by mapping all transmit channels combined (block 200). In particular, a single-mode B1+ map scan is performed. The single-mode B1+ map scan is performed in circularly polarized (CP) mode or quadrature mode which generates a rotating RF magnetic field.

The method 196 further includes inputting the generated/acquired quadrature B1+ maps into a deep learning-based radio frequency shimming inference model (block 202). The radio frequency shimming inference model is configured to infer RF shims based on inputted quadrature B1+ maps. The method 196 even further includes outputting from the deep learning-based radio frequency shimming inference model radio frequency shims based on the quadrature B1+ maps (block 204). The method 196 further includes applying the radio frequency shims to a magnetic resonance imaging scanner prior to the parallel transmission diagnostic scan of the imaging volume of the subject (block 206). The method 196 further includes performing the parallel transmission diagnostic scan to acquire magnetic resonance scan data of the imaging volume of the subject (block 208). The method 196 even further includes generating a reconstructed image based on the magnetic resonance scan data (block 210).

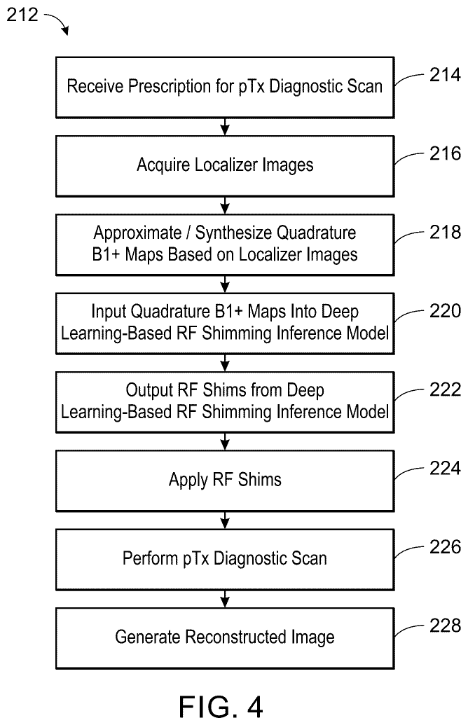

FIG. 4 illustrates a flow diagram of a method 212 for performing radio frequency (RF) shimming (e.g., utilizing localizer images). One or more steps of the method 212 may be performed by processing circuitry of the magnetic resonance imaging system 100 in FIG. 1 or a remote computing device. One or more of the steps of the method 212 may be performed simultaneously and/or in a different order from that depicted in FIG. 4. The method 212 is most beneficial for ultrahigh field MRI scanners (5 T, 7 T, and above) that are all equipped with at least 8-channel parallel transmission system. However, the method 212 can also be used for 3 T MRI scanners and above having at least two transmit channels.

The method 212 includes receiving a prescription for the parallel transmission diagnostic scan of the imaging volume of the subject (block 214). The prescription is prescribed via user input from a user or operator. The method 212 also includes, after receiving the prescription, acquiring localizer images of the imaging volume of the subject utilizing the magnetic resonance imaging scanner (block 216). The method 212 further includes approximating or synthesizing the quadrature B1+ maps based on the localizer images where mapping of all of the transmit channels is combined. (block 218)

The method 212 further includes inputting the approximate/synthesized quadrature B1+ maps into a deep learning-based radio frequency shimming inference model (block 220). The radio frequency shimming inference model is configured to infer RF shims based on inputted quadrature B1+ maps. The method 212 even further includes outputting from the deep learning-based radio frequency shimming inference model radio frequency shims based on the quadrature B1+ maps (block 222). The method 212 further includes applying the radio frequency shims to a magnetic resonance imaging scanner prior to the parallel transmission diagnostic scan of the imaging volume of the subject (block 224). The method 212 further includes performing the parallel transmission diagnostic scan to acquire magnetic resonance scan data of the imaging volume of the subject (block 226). The method 212 even further includes generating a reconstructed image based on the magnetic resonance scan data (block 228).

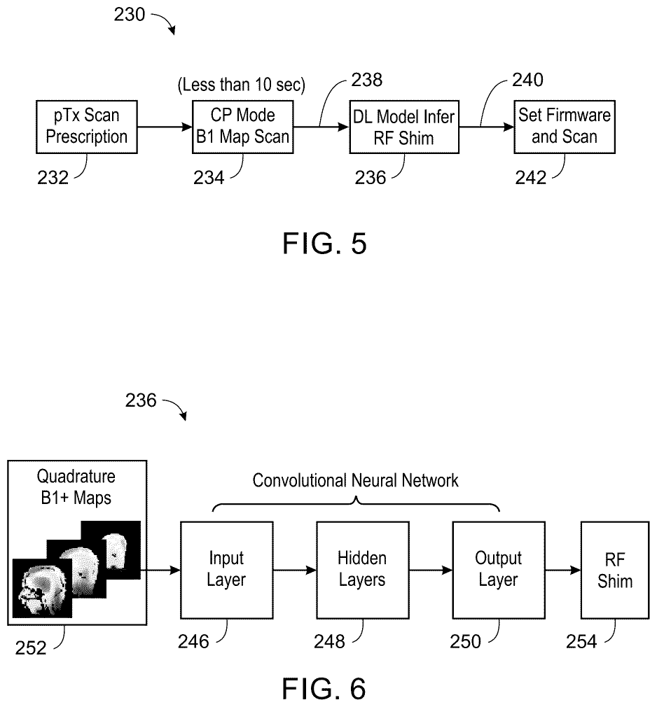

FIG. 5 illustrates a schematic diagram of a workflow 230 for performing radio frequency shimming. The workflow 230 includes receiving a prescription for the parallel transmission diagnostic scan of the imaging volume of the subject as indicated by reference numeral 232. The workflow 230 also includes, after receiving the prescription, performing a single-mode B1+ map scan (e.g., for less than ten seconds) in CP or quadrature mode (as part of a parallel transmission calibration scan) to generate quadrature B1+ maps by mapping all of the transmit channels combined as indicated by reference numeral 234. The workflow 230 further includes inputting the generated/acquired quadrature B1+ maps into a deep learning-based radio frequency shimming inference model 236 as indicated by arrow 238. The radio frequency shimming inference model 236 is configured to infer RF shims based on inputted quadrature B1+ maps. The workflow 230 even further includes outputting from the deep learning-based radio frequency shimming inference model 236 radio frequency shims based on the quadrature B1+ maps as indicated by arrow 240. The workflow 230 further includes applying the radio frequency shims to a magnetic resonance imaging scanner prior to the parallel transmission diagnostic scan of the imaging volume of the subject (by setting the firmware) and then performing the parallel transmission diagnostic scan to acquire magnetic resonance scan data of the imaging volume of the subject as indicated by reference numeral 242.

FIG. 6 illustrates a schematic diagram illustrating utilization of a trained neural network 236 (e.g., deep learning-based radio frequency shimming inference model) for performing radio frequency shimming. As depicted, the trained neural network 236 includes a convolutional neural network 244. In certain embodiments, the type of neural network may vary for the trained neural network 236 may vary. As depicted, the trained neural network 236 includes node layers including an input layer 246, hidden layers 248, and output layers 250. As depicted, quadrature B1+ maps 252 are inputted into trained neural network 236. The trained neural network 236 infers and outputs RF shims 254 (e.g., RF shim values) based on the inputted quadrature B1+ maps 252. In certain embodiments, supervised learning was utilized to train a neural network to generate the trained neural network 236. In certain embodiments, unsupervised learning may be utilized to generate the trained neural network 236.

An embodiment of a convolutional neural network based inference model may include multiple layers including but not limited to convolutional layers, rectified linear unit (ReLU) function layers, max pooling layers, dropout layers, flattening layers, and fully connected (FC) layers. The complexity of the model can be adjusted by changing the numbers, parameters, and combinations of these layers. An optimal model architecture can be tuned according to the number of training samples available, input image sizes, RF shim characteristics, and so on.

The deep learning-based radio frequency shimming inference model 236 (as utilized herein) was trained on 7 T B1+ maps from scans of brains of different subjects. The B1+ maps included multi-slices, multi-orientations, partial brain coverage, and whole brain coverage. The data was subject to preprocessing (e.g., normalization and/or masking). The acquired B1+ maps were subjected to augmentation (e.g. rotation and/or perturbation) to generate additional data. The deep learning-based radio frequency shimming inference model 236 was trained with approximately 10,000 parameters utilizing supervised learning.

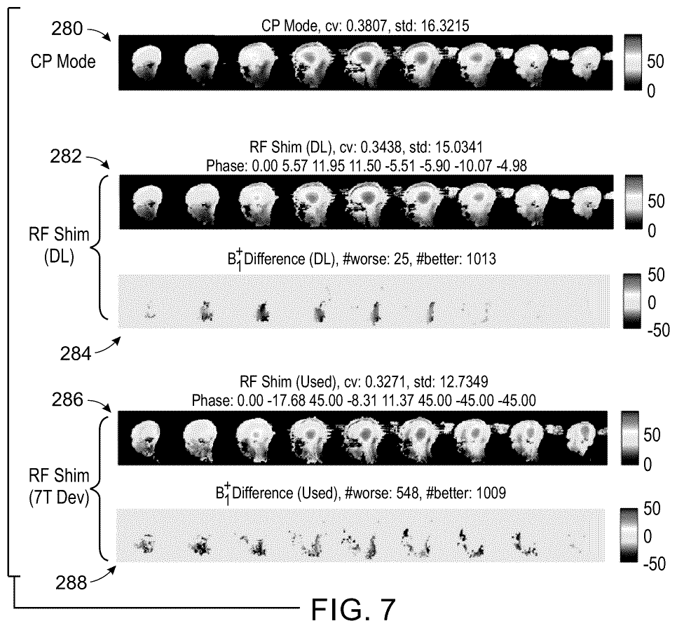

FIGS. 7-10 illustrate a performance of the deep learning-based radio frequency shimming inference model. FIG. 7 depicts the performance of the deep learning-based radio frequency shimming inference model when B1+ maps are corrupted by motion. A first row 280 depicts B1+ maps that were acquired in CP mode. A second row 282 depicts B1+ maps that were acquired after utilizing deep learning-based RF shimming (as described in the method 180 FIG. 2). A third row 284 depicts B1+ differences between the corresponding B1+ maps in the first row 280 and the first row 280. A fourth row 286 depicts B1+ maps that were acquired after utilizing algorithms for RF shimming utilized in a 7 T scanner. A fifth row 288 depicts B1+ differences between the corresponding B1+ maps in the fourth row 286 and the first row 280. As depicted, the trained deep learning-based radio frequency shimming inference model is motion insensitive and produces a more reliable RF shim when B1+ maps are corrupted by motion (compared to the algorithms for RF shimming utilized in a 7 T scanner). In particular, per-channel B1+ map data was corrupted by motion, which caused conventional RF shim optimization to produce suboptimal shims with new B1+ nulls. In contrast, the trained deep learning-based radio frequency shimming inference model predicted a more reliable RF shim, yielding significant B1+ improvements while minimizing any B1+ decreases.

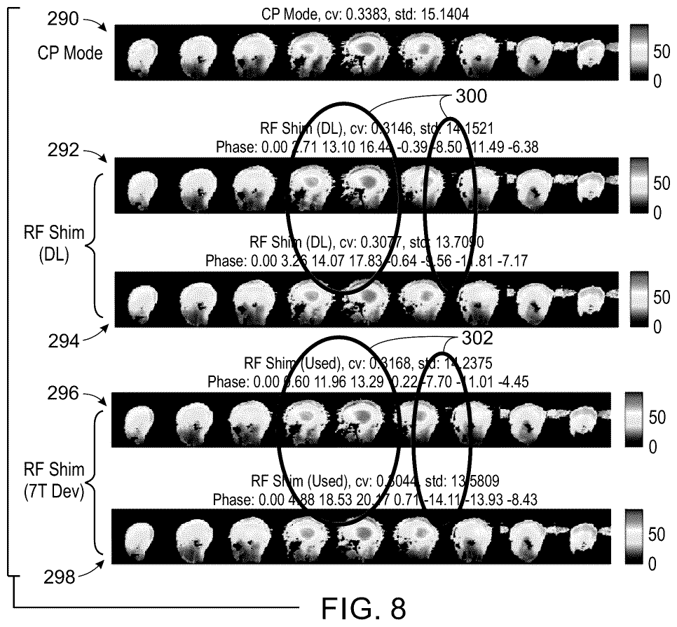

FIG. 8 depicts the performance of the deep learning-based radio frequency shimming inference model in a repeated RF shim scan. A first row 290 depicts B1+ maps that were acquired in CP mode. A second row 292 depicts B1+ maps that were acquired after utilizing deep learning-based RF shimming (as described in the method 180 FIG. 2). A third row 294 depicts B1+ maps that were acquired after utilizing deep learning-based RF shimming (as described in the method 180 FIG. 2) in a repeated RF shim scan. A fourth row 296 depicts B1+ maps that were acquired after utilizing algorithms for RF shimming utilized in a 7 T scanner. A fifth row 298 depicts B1+ maps that were acquired in a repeated RF shim scan after utilizing algorithms for RF shimming utilized in a 7 T scanner. As depicted, the trained deep learning-based radio frequency shimming inference model shows less variations in a repeated RF shim scan (compared to the algorithms for RF shimming utilized in a 7 T scanner when comparing like regions as indicated by ellipses 300 and 302) and thus provides a more robust RF shim performance.

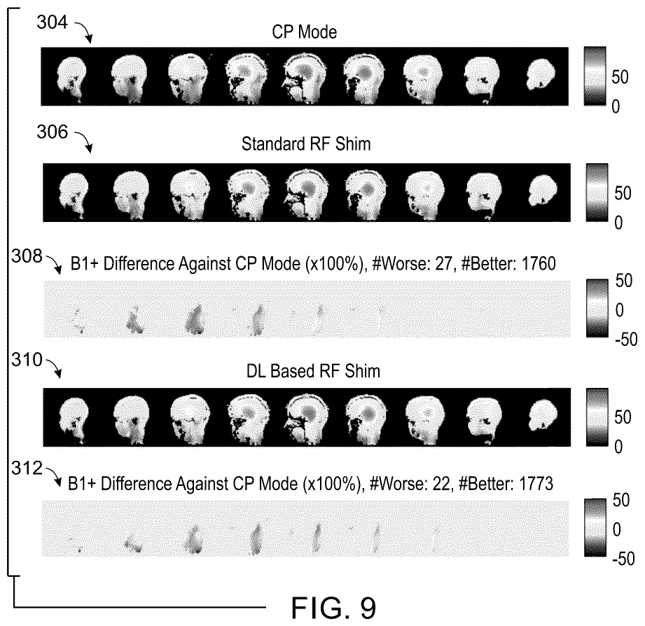

FIG. 9 illustrates a performance of the deep learning-based radio frequency shimming inference model with respect to low B1+ regions. A first row 304 depicts B1+ maps that were acquired in CP mode. A second row 306 depicts B1+ maps that were acquired after utilizing standard RF shimming (i.e., algorithm that reduces overall B1 non-uniformity with an emphasis on minimizing the shading area in the imaged field of view while maintaining a high transmit efficiency subjects to the limits of per-channel amplitude and phase changes). A third row 308 depicts B1+ differences between the corresponding B1+ maps in the first row 304 and the second row 306. A fourth row 310 depicts B1+ maps that were acquired after utilizing deep learning-based RF shimming (as described in the method 180 FIG. 2). A fifth row 312 depicts B1+ differences between the corresponding B1+ maps in the first row 304 and the fourth row 310. The B1+ maps with standard RF shimming and deep learning-based RF shimming produced overall similar B1+ changes with minor differences.

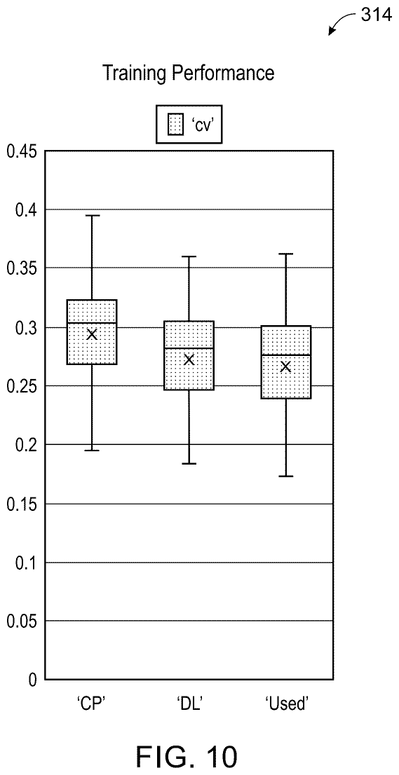

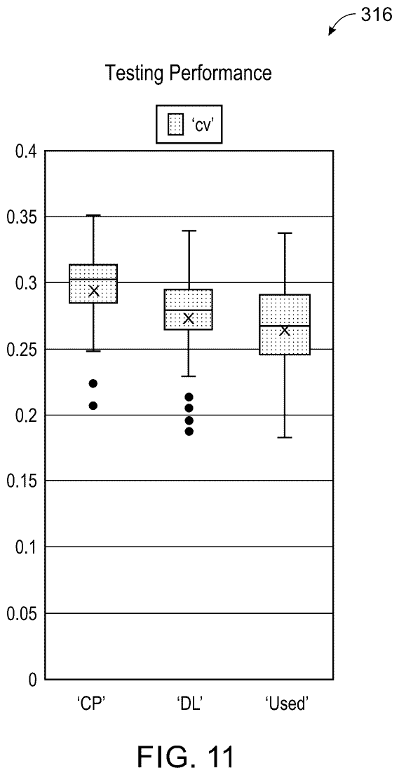

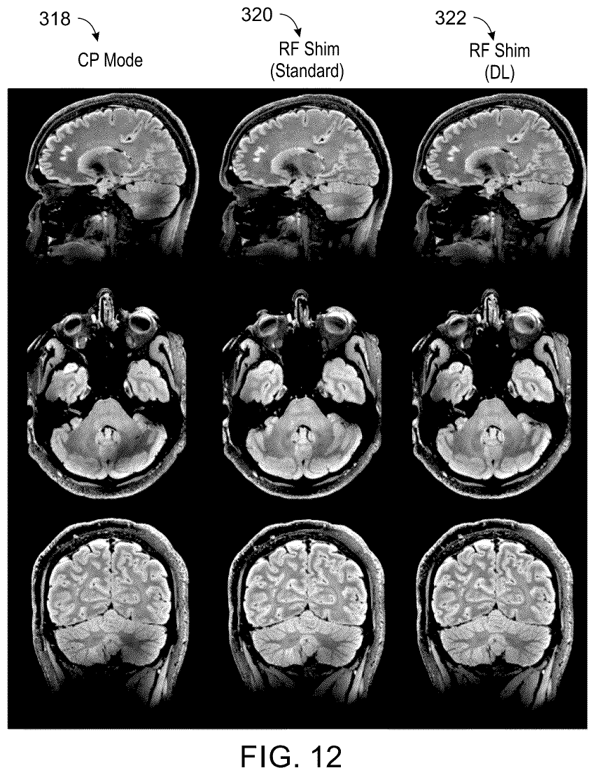

FIG. 10 illustrates a training performance of the deep learning-based radio frequency shimming inference model (e.g., on a training dataset). FIG. 11 illustrates a testing performance of the deep learning-based radio frequency shimming inference model (e.g., on a testing dataset). Subjects were scanned using B1+ sensitive sequences (e.g., fast spin echo, fluid-attenuated inversion recovery) utilizing a 7 T scanner with an 8Tx 32Rx head coil to evaluate the model's learning and generalization performance. Whole-brain per-channel B1+ map data was collected in axial, coronal, and sagittal planes used a saturation-prepared GRE method. To compare the performance between standard RF shimming and deep learning-based RF shimming, the B1+ maps of two RF shims were calculated, normalized, and compared against quadrature mode (CP mode). The coefficient of variation (CV) is calculated to evaluate RF shim quality and compared with quadrature (CP) mode.

FIG. 10 depicts a boxplot 314 comparing the CV of B1+ maps with the deep learning-based RF shimming (DL) and the standard RF shimming (Used) from training datasets. FIG. 11 depicts a boxplot 316 comparing the CV of B1+ maps with the deep learning-based RF shimming (DL) and the standard RF shimming (Used) from testing datasets. As depicted in FIGS. 10 and 11, the deep learning-based RF shimming produced lower CV for both training and testing datasets, respectively, suggesting good training and generalized performance of the deep learning-based radio frequency shimming inference model. The deep learning predicted RF shims are overall slightly more conservative than the standard approach as shown by the narrower extent of reduction in CV in the boxplot 314. In boxplot 316, lower CV values for both deep learning-based RF shimming and standard RF shimming are seen suggesting good, generalized capabilities.

FIG. 12 depicts MR images demonstrating a performance of the deep learning-based radio frequency shimming inference model on a previously seen subject. The M R images are three-dimensional (3D) T2-weighted FLAIR images. A first column 318 depicts MR images (sagittal, axial, and coronal MR images) acquired in CP mode of a previously seen subject. A second column 320 depicts MR images (sagittal, axial, and coronal MR images) acquired utilizing standard RF shimming of the same previously seen subject. A third column 322 depicts MR images (sagittal, axial, and coronal MR images) acquired utilizing deep learning-based RF shimming of the same previously seen subject. Shaded areas in the CP mode are mitigated after RF shimming with the deep learning-based RF shimming performing similarly to standard RF shimming.

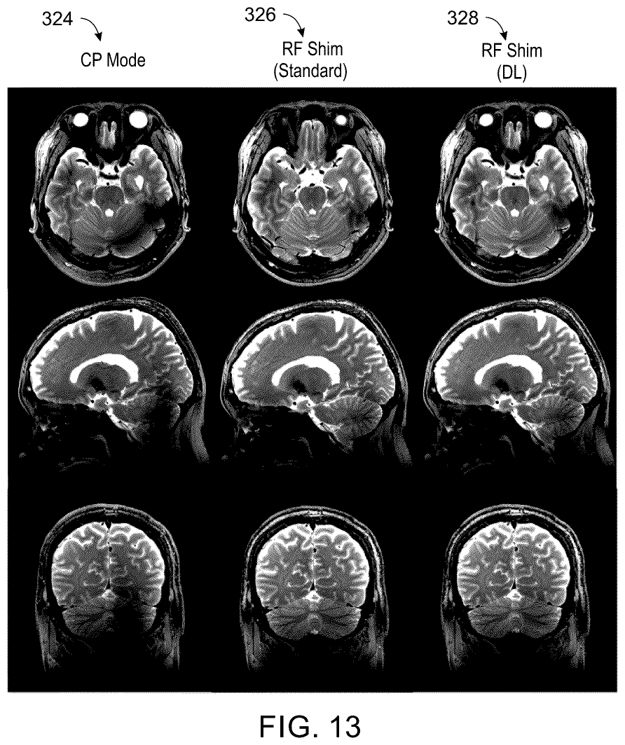

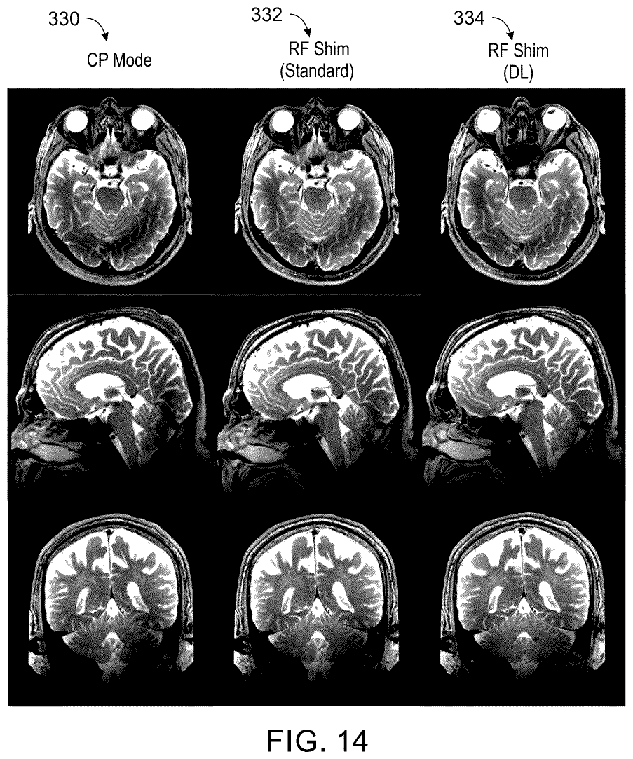

FIG. 13 depicts MR images demonstrating a performance of the deep learning-based radio frequency shimming inference model on a previously unseen subject. The MR images are two-dimensional (2D) T2-weighted FSE images. A first column 324 depicts MR images (axial, sagittal, and coronal MR images) acquired in CP mode of a previously seen subject. A second column 326 depicts MR images (axial, sagittal, and coronal MR images) acquired utilizing standard RF shimming of the same previously unseen subject. FIG. 14 depicts MR images demonstrating a performance of the deep learning-based radio frequency shimming inference model on an additional previously unseen subject. The MR images are two-dimensional (2D) T2-weighted FSE images. A first column 324 depicts MR images (axial, sagittal, and coronal MR images) acquired in CP mode of a previously seen subject. A second column 326 depicts MR images (axial, sagittal, and coronal MR images) acquired utilizing standard RF shimming of the same previously unseen subject. Shaded areas in the CP mode are reduced after RF shimming with the deep learning-based RF shimming performing similarly to standard RF shimming (even though the subject is new to the trained model).

Technical effects of the disclosed subject matter include providing systems and methods for performing deep learning-based radio frequency shimming. Technical effects of the disclosed subject matter include providing a faster and more robust RF shimming workflow to mitigate B1+ non-uniformities at ultrahigh field and improve image quality. The deep learning based RF shimming provides a faster workflow for ultrahigh field imaging. B1+ calibration scan time is greatly reduced with less sensitivity to motion while providing more consistent improvements in image quality. Technical effects of the disclosed subject matter include yielding a more consistent and robust RF shim that helps deliver higher quality images more reliably.

The disclosure also provides support for a computer-implemented method for performing radio frequency shimming, comprising: obtaining, via a processing system comprising one or more processors, quadrature B1+ maps of an imaging volume of a subject; inputting, via the processing system, the quadrature B1+ maps into a deep learning-based radio frequency shimming inference model; outputting, via the processing system, from the deep learning-based radio frequency shimming inference model radio frequency shims based on the quadrature B1+ maps; and applying, via the processing system, the radio frequency shims to a magnetic resonance imaging scanner prior to a parallel transmission diagnostic scan of the imaging volume of the subject. In a first example of the computer-implemented method, the computer-implemented method further comprises receiving, via the processing system, a prescription for the parallel transmission diagnostic scan of the imaging volume of the subject. In a second example of the computer-implemented method, optionally including the first example, the computer-implemented method further comprises, after receiving the prescription, performing, via the processing system, a parallel transmission calibration scan of the imaging volume of the subject with the magnetic resonance imaging scanner to obtain the quadrature B1+ maps by mapping all transmit channels combined. In a third example of the computer-implemented method, optionally including one or both of the first and second examples, the computer-implemented method further comprises, after receiving the prescription: acquiring, via the processing system, localizer images of the imaging volume of the subject utilizing the magnetic resonance imaging scanner; and synthesizing, via the processing system, the quadrature B1+ maps based on the localizer images. In a fourth example of the computer-implemented method, optionally including one or more or each of the first through third examples, the computer-implemented method further comprising performing, via the processing system, the parallel transmission diagnostic scan to acquire magnetic resonance scan data of the imaging volume of the subject. In a fifth example of the computer-implemented method, optionally including one or more or each of the first through fourth examples, the computer-implemented method further comprises generating, via the processing system, a reconstructed image based on the magnetic resonance scan data. In a sixth example of the computer-implemented method, optionally including one or more or each of the first through fifth examples, the magnetic resonance imaging scanner comprises at least two transmit channels and the magnetic resonance imaging scanner has a magnetic field strength of at least 3 Tesla.

The disclosure also provides support for a system performing radio frequency shimming, comprising: a memory encoding processor-executable routines; and a processing system comprising one or more processors and configured to access the memory and to execute the processor-executable routines, wherein the processor-executable routines, when executed by the processing system, cause the processing system to: obtain quadrature B1+ maps of an imaging volume of a subject; input the quadrature B1+ maps into a deep learning-based radio frequency shimming inference model; output from the deep learning-based radio frequency shimming inference model radio frequency shims based on the quadrature B1+ maps; and apply the radio frequency shims to a magnetic resonance imaging scanner prior to a parallel transmission diagnostic scan of the imaging volume of the subject. In a first example of the system, the processor-executable routines, when executed by the processing system, further cause the processing system to receive a prescription for the parallel transmission diagnostic scan of the imaging volume of the subject. In a second example of the system, optionally including the first example, the processor-executable routines, when executed by the processing system, further cause the processing system, after receiving the prescription, to perform a parallel transmission calibration scan of the imaging volume of the subject with the magnetic resonance imaging scanner to obtain the quadrature B1+ maps by mapping all transmit channels combined. In a third example of the system, optionally including one or both of the first and second examples, the processor-executable routines, when executed by the processing system, further cause the processing system, after receiving the prescription, to: acquire localizer images of the imaging volume of the subject utilizing the magnetic resonance imaging scanner; and synthesize the quadrature B1+ maps based on the localizer images. In a fourth example of the system, optionally including one or more or each of the first through third examples, the processor-executable routines, when executed by the processing system, further cause the processing system to perform the parallel transmission diagnostic scan to acquire magnetic resonance scan data of the imaging volume of the subject. In a fifth example of the system, optionally including one or more or each of the first through fourth examples, the processor-executable routines, when executed by the processing system, further cause the processing system to generate a reconstructed image based on the magnetic resonance scan data. In a sixth example of the system, optionally including one or more or each of the first through fifth examples, the magnetic resonance imaging scanner comprises at least two transmit channels and the magnetic resonance imaging scanner has a magnetic field strength of at least 3 Tesla.

The disclosure also provides support for a non-transitory computer-readable medium, the computer-readable medium comprising processor-executable code that when executed by a processing system comprising one or more processors, causes the processing system to: obtain quadrature B1+ maps of an imaging volume of a subject; input the quadrature B1+ maps into a deep learning-based radio frequency shimming inference model; output from the deep learning-based radio frequency shimming inference model radio frequency shims based on the quadrature B1+ maps; and apply the radio frequency shims to a magnetic resonance imaging scanner prior to a parallel transmission diagnostic scan of the imaging volume of the subject. In a first example of the non-transitory computer-readable medium, the processor-executable code, when executed by the processing system, further causes the processing system to receive a prescription for the parallel transmission diagnostic scan of the imaging volume of the subject. In a second example of the non-transitory computer-readable medium, optionally including the first example, the processor-executable code, when executed by the processing system, further causes the processing system, after receiving the prescription, to perform a parallel transmission calibration scan of the imaging volume of the subject with the magnetic resonance imaging scanner to obtain the quadrature B1+ maps by mapping all transmit channels combined. In a third example of the non-transitory computer-readable medium, optionally including one or both of the first and second examples, the processor-executable code, when executed by the processing system, further causes the processing system, after receiving the prescription, to: acquire localizer images of the imaging volume of the subject utilizing the magnetic resonance imaging scanner; and synthesize the quadrature B1+ maps based on the localizer images. In a fourth example of the non-transitory computer-readable medium, optionally including one or more or each of the first through third examples, the processor-executable code, when executed by the processing system, further causes the processing system to: perform the parallel transmission diagnostic scan to acquire magnetic resonance scan data of the imaging volume of the subject; and generate a reconstructed image based on the magnetic resonance scan data. In a fifth example of the non-transitory computer-readable medium, optionally including one or more or each of the first through fourth examples, the magnetic resonance imaging scanner comprises at least two transmit channels and the magnetic resonance imaging scanner has a magnetic field strength of at least 3 Tesla.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform]ing [a function] . . . ”, it is intended that such elements are to be interpreted under 35 U.S.C. 112(f). However, for any claims containing elements designated in any other manner, it is intended that such elements are not to be interpreted under 35 U.S.C. 112(f).

This written description uses examples to disclose the present subject matter, including the best mode, and also to enable any person skilled in the art to practice the subject matter, including making and using any devices or systems and performing any incorporated methods. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A computer-implemented method for performing radio frequency shimming, comprising:
obtaining, via a processing system comprising one or more processors, quadrature B1+ maps of an imaging volume of a subject;
inputting, via the processing system, the quadrature B1+ maps into a deep learning-based radio frequency shimming inference model;
outputting, via the processing system, from the deep learning-based radio frequency shimming inference model radio frequency shims based on the quadrature B1+ maps; and
applying, via the processing system, the radio frequency shims to a magnetic resonance imaging scanner prior to a parallel transmission diagnostic scan of the imaging volume of the subject.

2. The computer-implemented method of claim 1, further comprising receiving, via the processing system, a prescription for the parallel transmission diagnostic scan of the imaging volume of the subject.

3. The computer-implemented method of claim 2, further comprising, after receiving the prescription, performing, via the processing system, a parallel transmission calibration scan of the imaging volume of the subject with the magnetic resonance imaging scanner to obtain the quadrature B1+ maps by mapping all transmit channels combined.

4. The computer-implemented method of claim 2, further comprising, after receiving the prescription:
acquiring, via the processing system, localizer images of the imaging volume of the subject utilizing the magnetic resonance imaging scanner; and
synthesizing, via the processing system, the quadrature B1+ maps based on the localizer images.

5. The computer-implemented method of claim 1, further comprising performing, via the processing system, the parallel transmission diagnostic scan to acquire magnetic resonance scan data of the imaging volume of the subject.

6. The computer-implemented method of claim 5, further comprising generating, via the processing system, a reconstructed image based on the magnetic resonance scan data.

7. The computer-implemented method of claim 1, wherein the magnetic resonance imaging scanner comprises at least two transmit channels and the magnetic resonance imaging scanner has a magnetic field strength of at least 3 Tesla.

8. A system for performing radio frequency shimming, comprising:
a memory encoding processor-executable routines; and
a processing system comprising one or more processors and configured to access the memory and to execute the processor-executable routines, wherein the processor-executable routines, when executed by the processing system, cause the processing system to:
obtain quadrature B1+ maps of an imaging volume of a subject;
input the quadrature B1+ maps into a deep learning-based radio frequency shimming inference model;
output from the deep learning-based radio frequency shimming inference model radio frequency shims based on the quadrature B1+ maps; and
apply the radio frequency shims to a magnetic resonance imaging scanner prior to a parallel transmission diagnostic scan of the imaging volume of the subject.

9. The system of claim 8, wherein the processor-executable routines, when executed by the processing system, further cause the processing system to receive a prescription for the parallel transmission diagnostic scan of the imaging volume of the subject.

10. The system of claim 9, wherein the processor-executable routines, when executed by the processing system, further cause the processing system, after receiving the prescription, to perform a parallel transmission calibration scan of the imaging volume of the subject with the magnetic resonance imaging scanner to obtain the quadrature B1+ maps by mapping all transmit channels combined.

11. The system of claim 9, wherein the processor-executable routines, when executed by the processing system, further cause the processing system, after receiving the prescription, to:
acquire localizer images of the imaging volume of the subject utilizing the magnetic resonance imaging scanner; and
synthesize the quadrature B1+ maps based on the localizer images.

12. The system of claim 8, wherein the processor-executable routines, when executed by the processing system, further cause the processing system to perform the parallel transmission diagnostic scan to acquire magnetic resonance scan data of the imaging volume of the subject.

13. The system of claim 12, wherein the processor-executable routines, when executed by the processing system, further cause the processing system to generate a reconstructed image based on the magnetic resonance scan data.

14. The system of claim 8, wherein the magnetic resonance imaging scanner comprises at least two transmit channels and the magnetic resonance imaging scanner has a magnetic field strength of at least 3 Tesla.

15. A non-transitory computer-readable medium, the computer-readable medium comprising processor-executable code that when executed by a processing system comprising one or more processors, causes the processing system to:
obtain quadrature B1+ maps of an imaging volume of a subject;
input the quadrature B1+ maps into a deep learning-based radio frequency shimming inference model;
output from the deep learning-based radio frequency shimming inference model radio frequency shims based on the quadrature B1+ maps; and
apply the radio frequency shims to a magnetic resonance imaging scanner prior to a parallel transmission diagnostic scan of the imaging volume of the subject.

16. The non-transitory computer-readable medium of claim 15, wherein processor-executable code, when executed by a processing system, further causes the processing system to receive a prescription for the parallel transmission diagnostic scan of the imaging volume of the subject.

17. The non-transitory computer-readable medium of claim 16, wherein processor-executable code, when executed by a processing system, further causes the processing system, after receiving the prescription, to perform a parallel transmission calibration scan of the imaging volume of the subject with the magnetic resonance imaging scanner to obtain the quadrature B1+ maps by mapping all transmit channels combined.

18. The non-transitory computer-readable medium of claim 16, wherein processor-executable code, when executed by a processing system, further causes the processing system, after receiving the prescription, to:
acquire localizer images of the imaging volume of the subject utilizing the magnetic resonance imaging scanner; and
synthesize the quadrature B1+ maps based on the localizer images.

19. The non-transitory computer-readable medium of claim 15, wherein processor-executable code, when executed by a processing system, further causes the processing system to:
perform the parallel transmission diagnostic scan to acquire magnetic resonance scan data of the imaging volume of the subject; and
generate a reconstructed image based on the magnetic resonance scan data.

20. The non-transitory computer-readable medium of claim 15, wherein the magnetic resonance imaging scanner comprises at least two transmit channels and the magnetic resonance imaging scanner has a magnetic field strength of at least 3 Tesla.
